(19) United States
(12) Patent Application Publication
(10) Pub. No.: US 2025/0000934 A1
(43) Pub. Date: Jan. 2, 2025

(54) LYOPHILIZED SOVATELTIDE-BASED INJECTABLE FORMULATION AND METHOD FOR PREPARATION THEREOF

(71) Applicant: PHARMAZZ, INC., Willowbrook, IL (US)

(72) Inventors: Manish S. Lavhale, Uttar Pradesh (IN); Anil Gulati, Naperville, IL (US)

(21) Appl. No.: 18/478,528

(22) Filed: Sep. 29, 2023

Related U.S. Application Data
(63) Continuation-in-part of application No. 18/343,087, filed on Jun. 28, 2023.

(51) Int. Cl. A61K 38/10; A61K 9/19; A61K 47/12; A61K 47/26

(52) U.S. Cl. CPC A61K 38/10; A61K 9/19; A61K 47/12; A61K 47/26

(57) ABSTRACT

A lyophilized Sovateltide-based injectable formulation, comprising: i) an active pharmaceutical ingredient in the range of 0.01-0.02% w/w, ii) at least two soluble excipients in the range of 20-80% w/w, and iii) water for injection in the range of 1-2% w/w. A method for preparation of the lyophilized Sovateltide based injectable formulation comprises of following steps: i) dissolving Trisodium Citrate Dihydrate in water for injection to obtain a mixture, ii) adding active pharmaceutical ingredient in mixture, followed by addition of Mannitol to obtain a solution. iii) checking pH of solution, for making volume of solution, followed by sterile filtering the solution, and filling sterile filtered solution in vials, half stoppering of plugs on vials are, followed by loading vials in freeze dryer for lyophilization to obtain formulation, and v) unloading the vials are from freeze dryer and sealing of vials.

### CROSS-REFERENCE TO RELATED APPLICATION

This application is a continuation-in-part of U.S. Non-Provisional patent application Ser. No. 18/343,087, filed Jun. 28, 2023; the entire contents of which are incorporated by reference herein.

### FIELD OF THE INVENTION

The present invention relates to a lyophilized sovateltide-based injectable formulation and method for preparation thereof for treatment of various neurological diseases and disorders by providing intravenous route of administration and avoiding pain or irritation caused due to conventional procedures.

### BACKGROUND OF THE INVENTION

There are various diseases and disorders that are responsible for discomfort in life along with being fatal for life but have very limited therapeutic solutions. Few of such diseases/disorders includes neurological disorders or diseases including cerebral stroke, Alzheimer's disease, spinal cord injury, cognitive impairment, neurofibromatosis, Huntington's disease, Parkinson's, neonatal hypoxic-ischemic encephalopathy, multi-infarct dementia. Although various drugs have been discovered and synthesized for addressing such diseases and disorders. However, there are still concerns regarding their effectiveness and potential side effects.

Sovateltide is one such drug having therapeutic potential for treatment of such diseases/disorders, preferably neurological disorders. Sovateltide or succinyl-(glutamyl(9)-alanyl(11,15))-endothelin-1 (8-21) is a compound with 15 amino acid long chain. Sovateltide mimics the properties of a naturally occurring peptide called endothelin-1 (ET-1). ET-1 is known to activate two types of receptors in the body, namely endothelin A (ETARs) and endothelin B (ETBRs). Sovateltide is designed to specifically activate the ETBRs, making it a selective agonist for this receptor type. In fact, the aqueous solution of Sovateltide is susceptible to deterioration during storage.

RU2739382C1 discloses FIELD: medicine. SUBSTANCE: invention refers to treating stroke, particularly to a new method for treating stroke involving administration of Imatinib. Imatinib is administered to a patient in dose of 650 to 1600 mg/day on day 1 and in dose of 650 to 1200 mg/day for at least 2 successive consecutive days, preferably for at least 3 successive consecutive days and most preferably for at least 4 successive consecutive days. Imatinib administration for more than 3 days improves the neurological outcome in the post-stroke patients, as well as improves functional independence of the patient. EFFECT: invention improves the treatment of acute ischemic or haemorrhagic stroke, widens the therapeutic window for thrombolysis. RU'382 discloses about usage of Imatinib for treatment of stroke by administering first dose through intravenous route and subsequent doses through oral route. However, since Imatinib is administered through oral route in subsequent doses, the reaction time is more.

U.S. Pat. No. 8,361,459B2 discloses cells derived from postpartum tissue such as the umbilical cord and placenta, and methods for their use to regenerate, repair, and improve neural tissue, and to improve behavior and neurological function in stroke patients. US'459 mentions about treatment of stroke and other acute neural degenerative disorders by using postpartum-derived cells. However, the method mentioned in the prior art is very specific for stroke and acute neural degenerative disorders, and therefore have narrow range of application.

Conventionally, various methods are already known for treatment of stroke and other acute neural degenerative disorders. However, such methods either involves usage of multiple doses of drugs which have high reaction time or have narrow range of action.

In order to overcome the aforementioned drawbacks, there exist a need in the art to develop a Sovateltide-based formulation for treatment of various neurological diseases and disorders that is in lyophilized form in order to prevent deterioration of the formulation and capable of being administered through intravenous route in order to prevent any chances of irritation to the patient that generally occur in conventional treatment procedures.

### OBJECTS OF THE INVENTION

The principal object of the present invention is to overcome the disadvantages of the prior art.

An object of the present invention is to develop a formulation containing Sovateltide in a lyophilized form for treatment of various neurological diseases and disorders that exhibits greater inherent stability compared to the Sovateltide solution.

Another object of the present invention is to develop a formulation that is capable of being administered through intravenous route.

Another object of the present invention is to develop a formulation that is patient-compliant.

Yet another object of the present invention is to develop a formulation that contains Sovateltide in a very less amount, thereby preventing any chances of side-effects due to over-dosing of Sovateltide.

The foregoing and other objects, features, and advantages of the present invention will become readily apparent upon further review of the following detailed description of the preferred embodiment as illustrated in the accompanying drawings.

### SUMMARY OF THE INVENTION

The present invention relates to a lyophilized Sovateltide-based injectable formulation and method for preparation thereof that are administrated intravenously for treating various neurological disorders and diseases.

According to an embodiment of the present invention, a lyophilized Sovateltide-based injectable formulation, comprising: i) an active pharmaceutical ingredient in the range of 0.01-0.02% w/w, ii) at least two soluble excipients in the range of 20-80% w/w, and iii) water for injection in the range of 1-2% w/w.

According to another embodiment of the present invention, a method for preparation of the lyophilized Sovateltide-based injectable formulation comprises of following steps: i) Trisodium Citrate Dihydrate dissolved in the water for injection in order to obtain a mixture, ii) the active pharmaceutical ingredient-Sovateltide is added in the mixture, followed by addition of the mannitol in order to obtain a solution, iii) the pH of the solution is checked, for making the volume of the solution, followed by sterile filtering the solution and sterile filling the filtered solution in vials, and iv) the plugs on the vials are half stoppered, followed by loading the vials in freeze dryer for lyophilization to obtain the lyophilized Sovateltide-based injectable formulation.

While the invention has been described and shown with particular reference to the preferred embodiment, it will be apparent that variations might be possible that would fall within the scope of the present invention.

### BRIEF DESCRIPTION OF THE DRAWINGS

These and other features, aspects, and advantages of the present invention will become better understood with regard to the following description, appended claims, and accompanying drawings where:

FIG. 1 illustrates a flow chart of a process of developing lyophilized Sovateltide-based injectable formulation.

### DETAILED DESCRIPTION OF THE INVENTION

The following description includes the preferred best mode of one embodiment of the present invention. It will be clear from this description of the invention that the invention is not limited to these illustrated embodiments but that the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a,” “an,” and “the” designate both the singular and the plural, unless expressly stated to designate the singular only.

As used herein, the term “Pharmaceutical dosage forms” refer to specific forms or preparations in which drugs or pharmaceutical substances are formulated and administered to patients for therapeutic purposes. Such dosage forms may vary in their physical state, such as solid, liquid or semi-solid, and their route of administration, such as oral, topical, intravenous, or inhalation.

As used herein, the term “freeze-dried or lyophilized” powder or cake or preparation refers to any solid substance that has undergone the process of lyophilization or freeze-drying of aqueous solution. Ideally, a lyophilized preparation is obtained by freeze-drying a solution made up of aqueous solvents.

As used herein, the term “Stable pharmaceutical composition” refers to a formulation of a drug or active ingredient that maintains its physical, chemical, and therapeutic properties over a prolonged period, under appropriate storage conditions.

The present invention relates to a lyophilized Sovateltide-based injectable formulation and method for preparation thereof that is beneficial for the treatment of various neurological disorders and diseases without causing any side effects to the human body due to usage of less amount of Sovateltide in the formulation.

According to an embodiment of the present invention, a lyophilized Sovateltide-based injectable formulation, comprising: i) an active pharmaceutical ingredient in the range of 0.01-0.02% w/w, ii) at least two soluble excipient in the range of 20-80% w/w, and iii) water for injection in the range of 1-2% w/w.

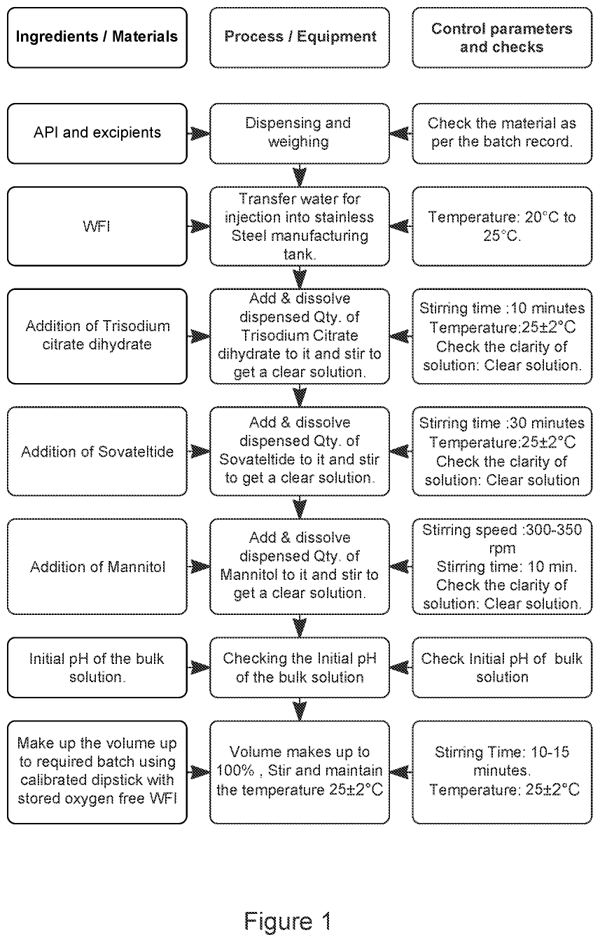

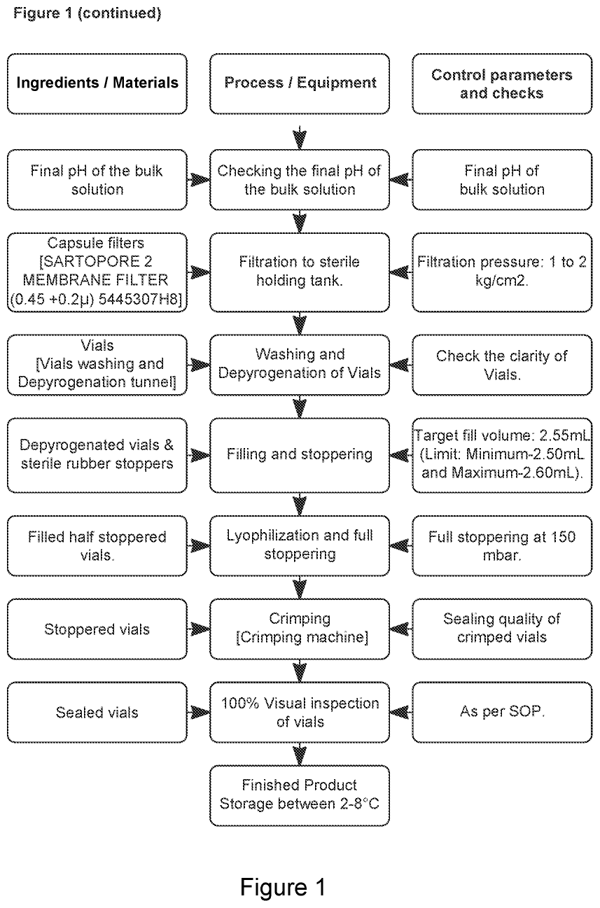

According to another embodiment of the present invention, a method for preparation of the lyophilized Sovateltide-based injectable formulation comprises of following steps: i) dissolving Trisodium Citrate Dihydrate in the water for injection in order to obtain a mixture, ii) adding the active pharmaceutical ingredient-Sovateltide in the mixture, followed by addition of the mannitol in order to obtain a solution, iii) checking the pH of the solution, for making the volume of the solution, followed by sterile filtering the solution and sterile filling the filtered solution in vials, and iv) half stoppering of plugs on the vials followed by loading the vials in freeze dryer for lyophilization to obtain the formulation (as illustrated in FIG. 1).

In an embodiment, the present invention comprises Sovateltide as an active ingredient, including a liquid injection having a solvent, that comprises at least one soluble excipient, wherein the soluble excipient includes mannitol, Trisodium citrate, Citric Acid anhydrous, Dibasic sodium citrate, Dibasic sodium Phosphate, Sodium Chloride, Hydroxypropyl cyclodextrin, where the soluble excipient serves multiple purposes of functioning as an osmolarity adjusting agent, pH modifier, and solubility enhancer.

In another embodiment, the osmolarity adjusting agent (buffering agent), pH modifier, and solubility enhancer are selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof.

In another embodiment, the freeze-drying filler is selected from the group consisting of sodium chloride, Trisodium citrate, Citric Acid anhydrous, Dibasic sodium citrate, Dibasic sodium Phosphate, Hydroxypropyl β-cyclodextrin, and mannitol and mixture thereof.

The presently disclosed subject matter is further illustrated by the following specific but not-limiting examples.

### EXAMPLE 1

The method for preparation of the lyophilized Sovateltide-based injectable formulation comprises of following steps: i) 4.800 gm of mannitol is dissolved in 75 ml of water for injection in order to obtain a mixture, ii) 0.990 mg of Sovateltide is added in the mixture, followed by addition of 1.500 gm of Trisodium Citrate Dihydrate in order to obtain a solution, iii) the pH of the solution is checked, for making the volume of the solution, followed by filtering the solution and filling the filtered solution in vials, and iv) the plugs on the vials are half stoppered, followed by loading the vials in freeze dryer for lyophilization to obtain the lyophilized Sovateltide-based injectable formulation. Table 1 represents the ingredients along with the composition used in the preparation of lyophilized Sovateltide-based injectable formulation.

TABLE 1
Ingredients along with the composition used in the preparation of
lyophilized Sovateltide-based injectable formulation (Example 1)

| Ingredient | Unit | Quantity used |
|---|---|---|
| Sovateltide | MG | 0.990 |
| Mannitol | GM | 4.800 |
| Trisodium Citrate | GM | 1.500 |
| Water for injection | ML | Q.S. to 75.0 |

### EXAMPLE 2

A method for preparation of the formulation of: i) 0.3 gm of mannitol is added in 30 ml of water for injection in order to obtain a mixture, ii) 0.990 mg of Sovateltide is added in the mixture, followed by addition of the 1% of Trisodium Citrate Dihydrate in order to obtain a solution, iii) the pH of the solution is checked and adjusted to 7.0, for making the volume of the solution, followed by filtering the solution and filling the filtered solution in vials, and iv) half stoppering of plugs on the vials, followed by loading the vials in freeze dryer for lyophilization to obtain the formulation. Table 2 represents the ingredients along with the composition used in the preparation of lyophilized Sovateltide-based injectable formulation.

TABLE 2
Ingredients along with the composition used in the preparation of
lyophilized Sovateltide-based injectable formulation (Example 2)

| Ingredient | Unit | Quantity used |
|---|---|---|
| Sovateltide | MG | 0.990 |
| Trisodium citrate | GM | 1% solution for pH adjustment |
| Mannitol | GM | 0.3 |
| Water for injection | ML | Q.S. to 30.0 |

### EXAMPLE 3

The method for preparation of the lyophilized Sovateltide-based injectable formulation is illustrated, which comprises of following steps: i) 0.3 gm of mannitol is added in 30 ml of water for injection in order to obtain a mixture, ii) 0.990 mg of Sovateltide is added in the mixture, followed by addition of 35 mg of 1% of Trisodium Citrate Dihydrate in order to obtain a solution, iii) the pH of the solution is checked and adjusted to pH 5.10 using 1% citric acid solution, for making the volume of the solution, followed by filtering the solution and filling the filtered solution in vials, and iv) half stoppering of plugs on the vials, followed by loading the vials in freeze dryer for lyophilization to obtain the formulation. Table 3 represents the ingredients along with the composition used in the preparation of lyophilized Sovateltide-based injectable formulation.

TABLE 3
Ingredients along with the composition used in the preparation of
lyophilized Sovateltide-based injectable formulation (Example 3)

| Ingredient | Unit | Quantity used |
|---|---|---|
| Sovateltide | MG | 0.990 |
| Trisodium citrate | MG | 35 |
| Mannitol | GM | 0.3 |
| Citric Acid Anhydrous | GM | 1% solution for pH adjustment |
| Water for injection | ML | Q.S. to 30.0 |

### EXAMPLE 4

The method for preparation of the lyophilized Sovateltide-based injectable formulation comprises of following steps: i) 0.3 gm of mannitol is added in 30 ml of water for injection in order to obtain a mixture, ii) 0.990 mg of Sovateltide is added in the mixture, and dissolve using 0.1% Dibasic sodium Phosphate solution in order to obtain a solution, iii) the pH of the solution is checked and adjusted to pH 7.51, for making the volume of the solution, followed by filtering the solution and filling the filtered solution in vials, and iv) half stoppering of plugs on the vials, followed by loading the vials in freeze dryer for lyophilization to obtain the formulation. Table 4 represents the ingredients along with the composition used in the preparation of lyophilized Sovateltide-based injectable formulation.

TABLE 4
Ingredients along with the composition used in the preparation of
lyophilized Sovateltide-based injectable formulation (Example 4)

| Ingredient | Unit | Quantity used |
|---|---|---|
| Sovateltide | MG | 0.990 |
| Dibasic sodium citrate | GM | 1% solution for pH adjustment |
| Mannitol | GM | 0.3 |
| Water for injection | ML | Q.S. to 30.0 |

### EXAMPLE 5

The method for preparation of the lyophilized Sovateltide-based injectable formulation comprises of following steps: i) 0.3 gm of mannitol is added in 30 ml of water for injection in order to obtain a mixture, ii) 0.990 mg of Sovateltide is added in the mixture, and dissolve using 9.0 mg Dibasic sodium Phosphate solution in order to obtain a solution, iii) the pH of the solution is checked and adjusted to pH 6.47, using 0.1% citric acid solution for making the volume of the solution, followed by filtering the solution and filling the filtered solution in vials, and iv) half stoppering of plugs on the vials, followed by loading the vials in freeze dryer for lyophilization to obtain the formulation. Table 5 represents the ingredients along with the composition used in the preparation of lyophilized Sovateltide-based injectable formulation.

TABLE 5
Ingredients along with the composition used in the preparation of
lyophilized Sovateltide-based injectable formulation (Example 5)

| Ingredient | Unit | Quantity used |
|---|---|---|
| Sovateltide | MG | 0.990 |
| Dibasic sodium Phosphate | MG | 9.0 |
| Mannitol | GM | 0.3 |
| Citric Acid Anhydrous | GM | 0.1% solution for pH adjustment |
| Water for injection | ML | Q.S. to 30.0 |

### EXAMPLE 6

The method for preparation of the lyophilized Sovateltide-based injectable formulation comprises of following steps: i) 0.6 gm of mannitol and 60.0 mg of Sodium chloride in 30 ml of water for injection in order to obtain a mixture, ii) 1.980 mg of Sovateltide is added in the mixture, and dissolved using 25.0 mg of Sodium Sulphate in order to obtain a solution, iii) the pH of the solution is checked and adjusted to pH 7.32, using 0.1% citric acid solution for making the volume of the solution, followed by filtering the solution and filling the filtered solution in vials, and iv) half stoppering of plugs on the vials, followed by loading the vials in freeze dryer for lyophilization to obtain the formulation. Table 6 represents the ingredients along with the composition used in the preparation of lyophilized Sovateltide-based injectable formulation.

TABLE 6
Ingredients along with the composition used in the preparation of
lyophilized Sovateltide-based injectable formulation (Example 6)

| Ingredient | Unit | Quantity used |
|---|---|---|
| Sovateltide | MG | 1.980 |
| Sodium Chloride | MG | 60.0 |
| Mannitol | GM | 0.6 |
| Sodium Sulphate | MG | 25.0 |
| Dibasic Sodium Phosphate | GM | 1% solution for pH adjustment |
| Water for injection | ML | Q.S. to 30.0 |

### EXAMPLE 7

The method for preparation of the lyophilized Sovateltide-based injectable formulation comprises of following steps: i) 0.150 gm of mannitol and 0.150 gm of Hydroxypropyl β-cyclodextrin is added in 30 ml of water for injection in order to obtain a mixture, ii) 0.990 mg of Sovateltide is added in the mixture, and dissolved using 1% of Trisodium Citrate in order to obtain a solution, iii) the pH of the solution is checked and adjusted to 7.10, using 1% solution of Trisodium Citrate Dihydrate for making the volume of the solution, followed by filtering the solution and filling the filtered solution in the vials, and iv) half stoppering of plugs on the vials, followed by loading the vials in freeze dryer for lyophilization to obtain the formulation. Table 7 represents the ingredients along with the composition used in the preparation of lyophilized Sovateltide-based injectable formulation.

TABLE 7
Ingredients along with the composition used in the preparation of
lyophilized Sovateltide-based injectable formulation (Example 7)

| Ingredient | Unit | Quantity used |
|---|---|---|
| Sovateltide | MG | 0.990 |
| Hydroxypropyl β-cyclodextrin | GM | 0.150 |
| Mannitol | GM | 0.150 |
| Trisodium Citrate | GM | 1% solution for pH adjustment |
| Water for injection | ML | Q.S. to 30.0 |

### EXAMPLE 8

The method for preparation of the lyophilized Sovateltide-based injectable formulation comprises of following steps: i) 0.150 gm of mannitol and 0.150 gm of Hydroxypropyl (3-cyclodextrin in 30 ml of water for injection in order to obtain a mixture, ii) 0.990 mg of Sovateltide is added in the mixture, and dissolved using 30 mg of Trisodium Citrate in order to obtain a solution, iii) the pH of the solution is checked and adjusted to pH 5.51 using 1% solution of Citric acid for making the volume of the solution, followed by filtering the solution and filling the filtered solution in vials, and iv) half stoppering of plugs on the vials, followed by loading the vials in freeze dryer for lyophilization to obtain the formulation. Table 8 represents the ingredients along with the composition used in the preparation of lyophilized Sovateltide-based injectable formulation.

TABLE 8
Ingredients along with the composition used in the preparation of
lyophilized Sovateltide-based injectable formulation (Example 8)

| Ingredient | Unit | Quantity used |
|---|---|---|
| Sovateltide | MG | 0.990 |
| Hydroxypropyl β-cyclodextrin | GM | 0.150 |
| Mannitol | GM | 0.150 |
| Citric acid Anhydrous | GM | 1% solution for pH adjustment |
| Trisodium Citrate | MG | 30.0 |
| Water for injection | ML | Q.S. to 30.0 |

### EXAMPLE 9

The method for preparation of the lyophilized Sovateltide-based injectable formulation is illustrated which comprises of following steps: i) 1.500 gm of mannitol is added in 30 ml of water for injection in order to obtain a mixture, ii) 0.990 mg of Sovateltide is added in the mixture, and dissolve by using 1% solution of Trisodium Citrate Dihydrate in order to obtain a solution, iii) the pH of the solution is checked and adjusted to pH 7.25 by using 1% solution of Trisodium Citrate Dihydrate for making the volume of the solution, followed by filtering the solution and filling the filtered solution in the vials, and iv) half stoppering of plugs on the vials, followed by loading the vials in freeze dryer for lyophilization to obtain the formulation. Table 9 represents the ingredients along with the composition used in the preparation of lyophilized Sovateltide-based injectable formulation.

TABLE 9
Ingredients along with the composition used in the preparation of
lyophilized Sovateltide-based injectable formulation (Example 9)

| Ingredient | Unit | Quantity used |
|---|---|---|
| Sovateltide | MG | 0.990 |
| Mannitol | GM | 1.500 |
| Trisodium Citrate | GM | 1% solution for pH adjustment |
| Water for injection | ML | Q.S. to 30.0 |

### EXAMPLE 10

The method for preparation of the lyophilized Sovateltide-based injectable formulation comprises of the following steps: i) 0.900 gm of Sodium Chloride is dissolved in 30 ml of water for injection in order to obtain a mixture, ii) 0.900 mg of Sovateltide is added in the mixture, and dissolved by using 1% solution of Trisodium Citrate Dihydrate followed by addition of 0.300 gm of Kollid PF-12 and stir to dissolve to obtain a solution, iii) the pH of the solution is checked and adjusted to pH 6.80 by using 1% solution of Trisodium Citrate Dihydrate for making the volume of the solution, followed by filtering the solution and filling the filtered solution in the vials, and iv) half stoppering of plugs on the vials are, followed by loading the vials in freeze dryer for lyophilization to obtain the formulation. Table 10 represents the ingredients along with the composition used in the preparation of lyophilized Sovateltide-based injectable formulation.

TABLE 10
Ingredients along with the composition used in the preparation of
lyophilized Sovateltide-based injectable formulation (Example 10)

| Ingredient | Unit | Quantity used |
|---|---|---|
| Sovateltide | MG | 0.990 |
| Sodium Chloride | GM | 0.900 |
| Kollidon PF-12 | GM | 0.300 |
| Trisodium Citrate | GM | 1% solution for pH adjustment |
| Water for injection | ML | Q.S. to 30.0 |

### EXAMPLE 11

The method for preparation of the lyophilized Sovateltide-based injectable formulation is illustrated which comprises of following steps: i) 1.500 gm of Mannitol is dissolved in 30 ml of water for injection in order to obtain a mixture, ii) 0.300 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 11 represents the ingredients along with the composition used in the preparation of lyophilized Sovateltide-based injectable formulation.

TABLE 11
Ingredients along with the composition used in the preparation of
lyophilized Sovateltide-based injectable formulation (Example 11)

| Ingredient | Unit | Quantity used |
|---|---|---|
| Sovateltide | — | — |
| Mannitol | GM | 1.500 |
| Trisodium Citrate | GM | 0.300 |
| Water for injection | ML | Q.S. to 30.0 |

### EXAMPLE 12

The method for preparation of the lyophilized Sovateltide-based injectable formulation which comprises of following steps: i) 1.500 gm of Mannitol is dissolved in 30 ml of water for injection in order to obtain a mixture, ii) 0.600 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 12 represents the ingredients along with the composition used in the preparation of lyophilized Sovateltide-based injectable formulation.

TABLE 12
Ingredients along with the composition used in the preparation of
lyophilized Sovateltide-based injectable formulation (Example 12)

| Ingredient | Unit | Quantity used |
|---|---|---|
| Sovateltide | — | — |
| Mannitol | GM | 1.500 |
| Trisodium Citrate | GM | 0.600 |
| Water for injection | ML | Q.S. to 30.0 |

### EXAMPLE 13

The method for preparation of the lyophilized Sovateltide-based injectable formulation comprises of following steps: i) 1.500 gm of Mannitol is dissolved in 30 ml of water for injection in order to obtain a mixture, ii) 0.990 mg of Sovateltide is added in the mixture and dissolved by using 0.900 gm of Trisodium Citrate Dihydrate to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 13 represents the ingredients along with the composition used in the preparation of lyophilized Sovateltide-based injectable formulation.

TABLE 13
Ingredients along with the composition used in the preparation of
lyophilized Sovateltide-based injectable formulation (Example 13)

| Ingredient | Unit | Quantity used |
|---|---|---|
| Sovateltide | MG | 0.990 |
| Mannitol | GM | 1.500 |
| Trisodium Citrate | GM | 0.900 |
| Water for injection | ML | Q.S. to 30.0 |

### EXAMPLE 14

The method for preparation of the lyophilized Sovateltide-based injectable formulation comprises of following steps: i) 7.500 gm of Mannitol is dissolved in 100 ml of water for injection in order to obtain a mixture, ii) 2.500 gm of Trisodium Citrate Dihydrate is added in the mixture to obtain the solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 14 represents the ingredients along with the composition used in the preparation of lyophilized Sovateltide-based injectable formulation.

TABLE 14
Ingredients along with the composition used in the preparation of
lyophilized Sovateltide-based injectable formulation (Example 14)

| Ingredient | Unit | Quantity used |
|---|---|---|
| Sovateltide | — | — |
| Mannitol | GM | 7.500 |
| Trisodium Citrate | GM | 2.500 |
| Water for injection | ML | Q.S. to 100.0 |

### EXAMPLE 15

The method for preparation of the lyophilized Sovateltide-based injectable formulation comprises of following steps: i) 7.500 gm of Mannitol is dissolved in 100 ml of water for injection in order to obtain a mixture, ii) 2.500 gm of Trisodium Citrate Dihydrate is added in the mixture and stirred to obtain a solution, iii) the pH of the solution is checked for making the volume of the solution, followed by filtering the solution and filling the filtered solution in the vials, and iv) half stoppering of plugs on the vials, followed by loading the vials in freeze dryer for lyophilization to obtain the formulation. Table 15 represents the ingredients along with the composition used in the preparation of lyophilized Sovateltide-based injectable formulation.

TABLE 15
Ingredients along with the composition used in the preparation of
lyophilized Sovateltide-based injectable formulation (Example 15)

| Ingredient | Unit | Quantity used |
|---|---|---|
| Sovateltide | — | — |
| Mannitol | GM | 7.500 |
| Trisodium Citrate | GM | 2.500 |
| Water for injection | ML | Q.S. to 100.0 |

### Results of Different Trails:

Referring to Table 16, a tabular representation of a outcomes of various experiments, which were conducted to investigate certain parameters is illustrated.

TABLE 16
Result of Trial

| | Parameters | | | | | |
|---|---|---|---|---|---|---|
| | | Clarity | | | | |
| | | & | | | | |
| | | Color of | Os- | | Parameter | |
| Trial | | recon- | molarity | | (Assay) | |
| Batch | Des- | stituted | (mOsmol/ | Assay | At | At |
| No. | cription | solution | Lit) | (2-8° C.) | (2-8° C.) | (25° C.) |
| Example | Complies | Complies | 266 | 100.97% | 100.61 | 100.51% |
| 1 | | | | | | |
| Example | Complies | Complies | Does not | 93.68% | — | 93.93% |
| 2 | | | comply | | | |
| Example | Complies | Complies | Does not | 90.85% | — | 86.19% |
| 3 | | | comply | | | |
| Example | Complies | Does not | Does not | 94.58% | — | 88.61% |
| 4 | | comply | comply | | | |
| Example | Complies | Does not | Does not | 98.43% | — | 84.81% |
| 5 | | comply | comply | | | |
| Example | Complies | Does not | Does not | 92.24% | — | 84.12% |
| 6 | | comply | comply | | | |
| Example | Complies | Does not | Does not | 104.88% | — | 100,22% |
| 7 | | comply | comply | | | |
| Example | Complies | Does not | Does not | 109.21% | — | 97.27% |
| 8 | | comply | comply | | | |
| Example | Complies | Complies | Does not | Not | — | Not done |
| 9 | | | comply | done | | |
| Example | Complies | Does not | Does not | Not | — | Not done |
| 10 | | comply | comply | done | | |
| Example | Complies | Complies | Does not | Placebo | — | Placebo |
| 11 | | | comply | | | |
| Example | Complies | Complies | Does not | Placebo | — | Placebo |
| 12 | | | comply | | | |
| Example | Complies | Complies | Does not | Not | — | Not done |
| 13 | | | comply | done | | |
| Example | Complies | Complies | 263 | Placebo | — | Placebo |
| 14 | | | | | | |
| Example | Complies | Complies | 275 | Placebo | — | Placebo |
| 15 | | | | | | |

The results of Example 1 were analyzed in accordance with the in-house specifications for Description, Clarity, Osmolarity and Assay, and found to meet these standards after one month of storage at a temperature of 25° C. As a result of these findings, pilot batches were produced for further testing and investigation.

Referring to FIG. 1, a flow chart of a process of developing an injectable formulation involves several steps are illustrated. The process of manufacturing Sovateltide injection 30 μg is explained in the flow chart.

### In Process Specification:

Referring to Table 17, a tabular representation of an outlines of specific parameters and requirements that must be met during the manufacturing of the formulation is illustrated. During the manufacturing process, various parameters are closely monitored to ensure that the final product meets the required standards. Among these, parameters are the description of the product, pH of the solution, assay, sterility, bacterial endotoxin test, and bioburden. After analysis, it was determined that the outcomes or results that were obtained fell within the predefined or specified range or limit.

TABLE 17
Outlines of specific parameters and requirements that must be
met during the manufacturing of the formulation

| | | |
|---|---|---|
| S. No. | Test | Specification |

Before Filtration

| | | |
|---|---|---|
| 1. | Description | Clear and Colorless solution |
| 2. | pH of solution | Between 7.0 to 8.5 |
| 3. | Bioburden | NMT 100 CFU/100 mL |

After Filtration

| | | |
|---|---|---|
| 1. | Description | Clear and Colorless solution |
| 2. | pH of solution | Between 7.0 to 8.5 |
| 3. | Assay (By | Not less than 90.0% w/w and not more |
| | HPLC) | than 110.0% w/w of the labelled |
| | | amount of Sovateltide |
| 4. | Sterility | The sample should be free from |
| | | microorganisms |
| 5. | Bacterial | Should not be more than 100 EU/mL |
| | endotoxin test | |
| 6. | Bioburden | Should not be more than |
| | | 10 CFU/100 mL |

### Determination of Freeze-Drying Cycles:

The vials filled with the filtered solution are subjected to lyophilization by loading in freeze dryer. The process involves three main stages: i) freezing, ii) primary drying, and iii) secondary drying. i) The primary drying stage is particularly critical and involves the sublimation of ice from the frozen product. In this case, the freeze-drying cycle to make the formulation lyophilized has been selected as follows. Firstly, the freezing stage is set to −45° C. This extremely low temperature ensures that the substance is frozen quickly and effectively. This step is essential to prevent the formation of large ice crystals that could damage the product's structure and composition.

After the freezing stage, ii) the primary drying stage begins. The temperature is set to −20° C. for specific duration. During this time, the ice crystals in the substance start to sublimate, turning from a solid state to a gaseous state. This process removes most of the water content from the product. iii) The next stage of primary drying is set to 0° C. for specific duration. This slower process ensures that all the remaining water content is removed from the product. Finally, the temperature is set to +10° C. for specific duration in the last stage of primary drying. At this temperature, any residual ice in the product sublimates, and the drying process is complete. The final product is a lyophilized formulation that can be stored for extended periods at 2° C.-8° C. or 22° C. to 28° C.

Employment of freeze-drying yields multiple benefits, which can be outlined as follows:
a) The present invention pertains to an injectable drug product possessing favorable attributes of physical appearance and rapid redissolution. The incorporation of mannitol in suitable proportions in the formulation has enabled the development of a facile and uncomplicated prescription with excellent flexibility, leading to the production of Sovateltide for injection. The addition of mannitol has facilitated the freezing of the solution, and the resultant dried frozen products exhibit enhanced solubility and clarity upon re-dissolution. Furthermore, the impurity content is low, and the stability of the product can be efficiently monitored, ensuring that the quality is well-regulated.
b) The product exhibits long-term stability and superior quality. One aspect of this embodiment pertains to freeze-dried formulations of Sovateltide. Further, the lyophilized preparation is packaged within a vial or another container that is appropriate for pharmaceutical use.

Thus, it may be concluded that the products derived from freeze drying process hold a notably low moisture content with a limit under 7.0% and can be stored at cold temperatures or at room temperature. The products exhibited no impurity content and maintained their original color as lyophilized injectable powder/cake. Upon redissolving, the resulting solution retains its clarity and exhibits no significant change. This overall demonstrates a high level of product stability. Bacterial endotoxin test result complies with the regulatory requirement, performed by Gel clot technique Limulus Amebocyte Lysate (LAL) reagent.

### Finished Product Specification:

Referring to Table 18, a tabular representation outlines the various parameters that have been established to ensure the quality and consistency of the finished product. The lyophilized sovateltide injection underwent an analysis of specified parameters, including its description, identification, reconstitution time, and appearance after reconstitution. The results of the analysis revealed that all of these parameters meet the required specifications, indicating that the product has passed the necessary quality standards. The reconstituted solution was found to be within the specified pH range of 7.0-8.5, with a pH value of 8.32. The moisture content, determined using the Karl Fischer method, was 2.61% w/w, well within the allowed limit of not more than 7%. Bacterial endotoxins were found to comply with regulatory requirements, and the amount of particulate matter for particles ≥10 μm and ≥25 μm were 81 and 02 per container, respectively, which were well within the limit. Specified impurity was not detected while unidentified impurity and total impurity was 0.39% and 0.87%, respectively. The assay value was observed to be 95.65% with osmolarity of 275 mOsm/L. These results of batch analysis confirm the success of the manufacturing process development. Lyophilized Sovateltide Injection, 30 μg should be stored between 2 to 8° C., protect from light.

TABLE 18
Outlines the various parameters that have been established to ensure the
quality and consistency of the finished product
(i.e. disclosed in EXAMPLE 1)

| S. No. | Test | Specification |
|---|---|---|
| 1. | Description | White to off-white lyophilized cake. |
| 2. | Reconstitution Time | Content should dissolve within 1 minute. |
| 3. | Clarity of | The solid should dissolve |
| | reconstituted | completely and the solution |
| | solution | should be visually clear. |
| 4. | Identification | |
| | A. By HPLC | The retention time of Sovateltide |
| | (By UV Detector) | peak in chromatogram of the test |
| | | sample should correspond |
| | | to that in the chromatogram |
| | | of the reference standards |
| | | as obtained in assay method. |
| | B. By HPLC | The PDA spectrum of |
| | (By PDA Detector) | Sovateltide in test sample |
| | | should correspond to that of |
| | | the reference standard as |
| | | obtained in assay method. |
| 5. | Particulate Matter | Particle ≥10 μm should |
| | | not exceed 6000 per |
| | | container. |
| | | Particle ≥25 μm should |
| | | not exceed 600 per |
| | | container. |
| 6. | pH | Between 7.0 to 8.5. |
| 7. | Moisture Content | Not more than 7.0%. |
| | (% w/w) | |
| 8. | Assay (%) | Not less than 90.0 and not more than |
| | | 110.0 of labelled amount of Sovateltide. |
| 9. | Related Substances | Specified Impurity |
| | (%) | D-His: NMT 1.0 |
| | | Unspecified Impurity: NMT 0.5 |
| | | Total Impurity: NMT 2.0 |
| 10. | Bacterial Endotoxin | Should not be more than 16666 EU/mg |
| | test | |
| 11. | Sterility Testing | Sample should be free |
| | | from microorganisms |
| 12. | Osmolarity (mOsm/L) | Between 240 to 310. |
| 13. | Uniformity of dosage | Maximum acceptance value |
| | unit (By Content | should be less than or |
| | Uniformity) | equal to 15.0. |

### Packaging Configuration:

The formulation in the vials was further packaged. The configurations of which are mentioned below:
Glass Vial—5 mL, USP type 1, Amber, tubular glass vial.
Rubber Stoper—13 mm Slotted Grey Bromo butyl “RFU” Rubber Stoppers.
Aluminum Seal—13 mm Aluminum Flip-Off Seal.

Referring to Table 19 and 20, a tabular representation of the specification and results of container and closure system are illustrated, respectively.

TABLE 19
The specifications for 13 mm Aluminum Flip-Off Tear-Off Seal

| Sr. No. | Description | Specification (mm) |
|---|---|---|
| 1 | Inner Diameter of Aluminum Seal | 13.00-13.60 |
| 2 | Outer Diameter of Aluminum Seal | 13.50-14.10 |
| 3 | Diameter of Plastic Disc | 14.50-15.00 |
| 4 | Height of Plastic Disc | 2.90 m-3.30 |
| 5 | Inner Height of Alum. Seal | 6.10-6.40 |
| 6 | Assembled Height | 7.62-8.38 |
| 7 | Thickness of Aluminium Cap | 0 18-0 20 |
| 8 | No. of Bridges | 06 Nos |
| 9 | Approved Drawing No | FO/VS/DE-13 |

As shown in Table 19, the packaging component Aluminum Flip-Off Tear-Off Seal (13 mm) was used with very precise measurements. The seal is designed to fit securely on a container with an inner diameter of 13.32 to 13.40 mm, while its outer diameter ranges from 13.78 to 13.90 mm. The plastic disc, which is an integral part of the seal and used to cover the top of the vial, has a diameter of 14.75 to 14.83 mm and a height of 3.02 to 3.10 mm. The inner height of the aluminum seal, which is the distance between the bottom of the seal and the top of the plastic disc, ranges between 6.25 mm to 6.32 mm. The assembled height of the seal, which is the distance between the bottom of the seal and the top of the plastic disc ranges between 7.81 mm to 8.02 mm. The thickness of the aluminum cap is 0.18 mm, which ensures that the seal is sturdy. Additionally, the seal complies with the approved drawing, ensuring that it meets all the necessary requirements.

TABLE 20
The specifications for 13 mm Grey Bromobutyl “RFU” Rubber stoppers

| S. No. | TESTS | LIMITS |
|---|---|---|
| 1. | Description | 13 mm slotted grey |
| | | coloured Bromobutyl |
| | | “RFU” rubber stoppers |
| 2. | Physical Parameter | |
| | Disc Diameter | 12.50 mm to 12.80 mm |
| | Flanges Thickness (Collar) | 02.00 mm to 02.40 mm |
| | Maximum Height | 10.20 mm to 10.80 mm |
| | Diameter | 07.70 mm to 08.10 mm |
| 3. | Fitment with vial | Should be satisfactory |
| 4. | Identification by IR | IR spectra of sample |
| | | should be concordant |
| | | with the standard spectra of |
| | | working/reference standard |
| | | of the rubber stopper |
| 5. | Total Ash | 41.40% to 50.60% |
| 6. | Appearance of solution | Solution A should not |
| | (Opalescence | be more opalescent |
| | and Colour) | than opalescence |
| | | standard OS3 and not |
| | | more intensely coloured |
| | | than reference |
| | | solution BYS6. |
| 7. | Acidity or Alkalinity | Not more than 0.3 ml of 0.01M |
| | | NaOH should be required |
| | | to produce blue colour |
| 8. | Light Absorption | Absorbance should |
| | | not be more than 2.0 |
| | | in the range of 220 to 360 nm |
| 9. | Reducing substance | The difference between the titration |
| | | volumes should not |
| | | be more than 7.0 ml |
| 10. | Heavy Metals | Not more than 20 ppm |
| 11. | Residue on Evaporation | Not more than 4.0 mg |
| 12. | Volatile sulphides | Black stains on paper |
| | | should not be more |
| | | intense than that of a standard. |
| 13. | Fragmentation Test | Total number of |
| | | fragments should not be |
| | | more than 15 |
| 14. | Sterilization test | The closures ‘prepared’ |
| | | should not soften |
| | | or become tacky and |
| | | there should be no |
| | | visual change in the closure |
| 15. | Liquid Particulate Test | Not more than 600 |
| 16. | | Not more than 60 |
| 17. | Sterility Test | No growth of micro-organism should |
| | | occur after 14 days of incubation. |
| 18. | Endotoxin Test | Not more than 0.25 EU/ml |

As shown in Table 20, that the 13 mm slotted grey colored Bromobutyl “RFU” rubber stoppers meet the requisite physical parameters. The diameter of the disc measures 12.68 mm, while the flanges thickness or collar measures 2.21 mm, and the maximum height is 10.47 mm. The diameter of the rubber stopper is 7.87 mm, and it demonstrates an optimal fitment with the vial. The IR spectra of the sample are concordant with the reference standard, enabling accurate identification of the rubber stopper. The appearance of solution A is consistent with acceptable opalescence and colour standards, with the solution demonstrating no more opalescence than opalescence standard OS3 and not more intensely coloured than reference solution BYS6. The rubber stopper manifests no significant acidity or alkalinity (0.19 mL) and is within the specified range, while light absorption (0.0028) and reducing substances (1.9 mL) are within the acceptable range. The heavy metal concentration is less than 20 ppm, and residue on evaporation is minimal i.e., 2.1 mg, indicating adherence to the desired parameters. The rubber stopper passed the sterilization test, demonstrating that it does not soften or become tacky, and there is no visual change in the closure. Further, the liquid particulate test results show that the number of particles that measure ≥10 μm is 17, while the number of particles >25 μm is 1, meeting the set specifications. The sterility test demonstrated that there was no growth observed, and the endotoxin test showed that the endotoxin concentration was less than 0.25 EU/ml, signifying compliance with the desired quality standards. Therefore, the 13 mm slotted grey coloured Bromobutyl “RFU” rubber stoppers have successfully met the requisite physical and chemical specifications, demonstrating their suitability for use.

Referring to Table 21, a tabular representation of a specification for Amber color vial is illustrated. The results present an exhaustive investigation of the physical parameters of a 5 ml amber color vial. The height of the vial was measured and was found to range from 48.00 mm to 48.73 mm, with an average height of 48.36 mm. Additionally, the rim height of the vial was observed to vary from 3.60 mm to 3.91 mm, with an average height of 3.75 mm. The diameter of the vial's body was found to range from 16.16 mm to 16.37 mm, with an average diameter of 16.27 mm. Furthermore, the mouth outer diameter of the vial was determined to vary from 12.94 mm to 13.02 mm, with an average diameter of 12.98 mm. Additionally, the mouth inner diameter of the vial was found to vary from 6.93 mm to 7.18 mm, with an average diameter of 7.05 mm. Furthermore, it was established that the T1 body wall thickness and T2 bottom wall thickness of the vial were both in compliance with the prescribed standards. The meticulous data collected from the analysis conclusively indicates that the 5 ml amber color vial is well within the specified range of parameters and meets the required standards. The vial has undergone rigorous visual testing and has been found to be in compliance with industry standards. The vial is made from borosilicate amber USP type glass that meets the requirements for construction. Furthermore, the product is free from dust, dirt, or any foreign particles that may affect its quality or function. In addition to this, the vial has also been evaluated for any chipping or bubbles that may compromise its integrity. As per the results, it complies with the set standards, indicating that it is free from any visible imperfections (Table 21). Furthermore, vial has been subjected to various tests including glass grain test, surface glass test, arsenic test and the results indicate compliance with the standards (Table 21).

TABLE 21
Specification for Amber color vial

| | |
|---|---|
| Parameters | Specification (mm) |

Analytical Report

| | |
|---|---|
| Total height (H1) | 48.00-49.00 |
| Rim height (H2) | 3.50-4.10 |
| Body diameter (D1) | 16.00-16.50 |
| Mouth outer diameter (D2) | 12.70-13.20 |
| Mouth inner diameter (D3) | 6.80-7.20 |
| Body wall thickness (T1) | NLT-0.80 |
| Bottom wall thickness (T2) | NLT-0.70 |

Visual Analysis

| | |
|---|---|
| Material of construction | Borosilicate amber USP type glass |
| Dust dirt or foreign particles | Absent |
| Chipping and bubbles | Absent |

Glass Quality

| | |
|---|---|
| Glass grain test | Maximum volume of |
| | 0.02M HCl/ g. of test glass |
| | should be not more than 0.1 mL. |
| Surface glass test | Maximum volume of 0.01M |
| | HCl. per 100 ml. of test |
| | liquid should be not more than 1.00 ml. |
| Arsenic test | Not more than 0.1 ppm |

### Stability Studies:

The stability of drug product-Sovateltide injection 30 μg stored in USP type I glass vial conducted at 25° C.±2° C. and 60% RH ±5% RH for not less than 12 months, wherein each vial contains 30 μg of Sovateltide. Initially the pH of the lyophilized injectable was 7.91 and after 12 months the pH was 7.67 and which is within the specified limit. The Reconstitution time, Clarity of reconstituted solution, moisture content, sterility, Bacterial Endotoxins limit, Particulate matter, Related Substances (%), all the parameters meet the regulatory requirements and are within the specified limit. Initially, the percent assay of lyophilized Sovateltide injection was 100.41% and after storage at 25° C.±2° C. and 60% RH±5% RH for not less than 12 months, the assay value was 98.81%. The results confirm that the formulation remains stable for not less than 12 months at a storage temperature of 25° C.±2° C.

Similarly, the developed pharmaceutical lyophilized injectable formulation of Sovateltide stored at a temperature ranging between 2° C.-8° C., is stable not less than 36 months of storage and the results complies with specified reconstitution time, clarity of reconstituted solution, moisture content, sterility, bacterial endotoxins limit, particulate matter, related substances (%). Initially, the pH of the lyophilized injectable stored at 5° C.±3° C. was 8.18 and after 36 months the pH was 7.77. Initially, percent assay of the lyophilized Sovateltide injection stored at 5±3° C. was 106.00% and after storage of formulation for not less than 36 months, the assay value was 95.58%. The results are in favor that the formulation remains stable for not less than 36 months at storage temperature of 5±3° C.

### Force Degradation Study:

Referring to Table 22, a tabular representation of a Force Degradation Study Sovateltide Injection is illustrated. Different conditions for forced degradation (chemical degradation) were set comprising 1 mL-5 N HCl/3 Hours RT; 1 mL-5 N NaOH/3 Hours RT; 1 mL-30% H₂O₂/3 Hours RT; for acid degradation, base degradation; and peroxide degradation, respectively. Different conditions for forced degradation (physical degradation) were set comprising 3 Hours/105° C.; 95% RH/24 Hours for Thermal Degradation and Humidity Degradation, respectively. Different conditions for forced degradation were set to observe photolytic degradation and the samples covered with Aluminum foil (Dark Condition); Final Packaging; Primary Packaging (Labelled); Primary Packaging (Unlabeled) and 1.2 million Lux hours & 200 Watt-Hour/m² (Open Condition). According to the findings, Lyophilized Sovateltide injection 30 μg demonstrated sensitivity to acid, base, peroxide, and photolytic degradation. The results of chromatography and peak purity analysis of the degraded samples revealed that the analyte peaks were uniform, with no co-eluting peaks from the degradation process. Sovateltide peak purity was maintained in all chromatograms under various stress conditions.

TABLE 22
Force Degradation Study Sovateltide Injection

| | | | | | | | | |
|---|---|---|---|---|---|---|---|---|
| | | Peak | Peak | | | | | |
| | | Purity | Purity | | Single | Total | | |
| | | Index | Index | Specified | Maximum | Unknown | Total | |
| | | of | of | impurity | Unknown | Impurities | Impurity | % |
| | | Sovateltide | Sovateltide | from | Impurity | from | (Method-1 + | Degradation |
| Degradation | Degradation | from | from | Method-2 | from | Method-1 | Method-2) | (w.r.t. |
| Mode | Condition | Method-1 | Method-2 | (%) | Method-1 (%) | (%) | (%) | Control) |

Chemical Degradation

| | | | | | | | | |
|---|---|---|---|---|---|---|---|---|
| Control | No | 0.999824 | 1.000000 | ND | 0.190 | 0.336 | 0.336 | — |
| | Treatment | | | | | | | |
| Acid | 1 mL-5N | 0.999992 | 0.999049 | ND | 2.937 | 3.195 | 3.195 | 2.86 |
| Degradation | HC;/ | | | | | | | |
| | 3 Hours | | | | | | | |
| | RT | | | | | | | |
| Base | 1 mL-5N | 1.000000 | 1.000000 | ND | 2.239 | 4.639 | 4.639 | 4.30 |
| Degradation | NaOH/ | | | | | | | |
| | 3 Hours | | | | | | | |
| | RT | | | | | | | |
| Peroxide | 1 mL- | 1.000000 | 1.000000 | ND | 1.840 | 6.830 | 6.830 | 6.49 |
| Degradation | 30% | | | | | | | |
| | H₂O₂/ 3 | | | | | | | |
| | Hours | | | | | | | |
| | RT | | | | | | | |

Physical Degradation

| | | | | | | | | |
|---|---|---|---|---|---|---|---|---|
| Thermal | 3 hours/ | 0.989942 | 1.000000 | ND | 1.357 | 5.199 | 5.199 | 4.86 |
| Degradation | 105° C. | | | | | | | |
| Humidity | 95% | 0.997178 | 1.000000 | ND | 0.280 | 0.740 | 0.740 | 0.40 |
| Degradation | RH/24 | | | | | | | |
| | Hours | | | | | | | |

Photolytic Degradation

| | | | | | | | | |
|---|---|---|---|---|---|---|---|---|
| Photolytic | Covered | 0.989942 | 1.000000 | ND | 1.485 | 1.964 | 1.964 | 1.63 |
| Degradation | with | | | | | | | |
| | Aluminium | | | | | | | |
| | foil | | | | | | | |
| | (Dark | | | | | | | |
| | Condition) | | | | | | | |
| | Final | 0.997178 | 0.999609 | ND | 0.249 | 0.846 | 0.846 | 0.51 |
| | Packaging | | | | | | | |
| | Primary | 1.000000 | 1.000000 | ND | 1.924 | 5.604 | 5.604 | 5.27 |
| | Packaging | | | | | | | |
| | (Labelled) | | | | | | | |
| | Primary | 1.000000 | 1.000000 | ND | 1.924 | 4.567 | 4.567 | 4.23 |
| | Packaging | | | | | | | |
| | (Unlabeled) | | | | | | | |
| | 1.2 | 0.999845 | 0.999998 | ND | 0.786 | 2.940 | 2.940 | 2.60 |
| | million | | | | | | | |
| | Lux | | | | | | | |
| | hours & | | | | | | | |
| | 200 | | | | | | | |
| | Watt- | | | | | | | |
| | Hour/m | | | | | | | |
| | 2 (Open | | | | | | | |
| | Condition) | | | | | | | |

### Method Validation for Assay of Sovateltide Injection:

Chromatographic Parameters for method validation of Assay of Sovateltide Injection: An analytical system comprising an Xtimate C18 (250×4.6 mm), 5 μm or Chromcore 120 C18 (250×4.6 mm), 5 μm or an analytical column that was equivalent was utilized for the experiment. The flow rate was maintained at 0.8 mL/min, while the injection volume was 80 μL. The detection wavelength was set at 215 nm, and the total run time was 30 minutes. The autosampler temperature was 5° C. and the column oven temperature was 40° C. The retention time was determined to be 13±2 minutes.

### Mobile Phase for Assay:

Mobile Phase-A, (10 mM KH₂PO₄ & 10 mM NaCl, pH—7.5

### Mobile Phase-B (Methanol)

Referring to Table 23, a tabular representation of a Method Validation Summary of Assay of Sovateltide Injection is illustrated. The method validation for the assay of Sovateltide in Sovateltide injection 30 μg was done by HPLC. After the evaluation of data, the method was found precise, linear, accurate, robust, rugged, and specific. As the results of all the validation parameters were within the acceptance criteria, it is concluded that the analytical method is suitable for the determination of Sovateltide by HPLC. Based on solution stability study, the Standard solution of Sovateltide was stable for up to 24 Hours at 5° C., sample solution of Sovateltide injection was stable for up to 26 Hours at 5° C. and sample solution of In-process bulk solution was found stable for up to 25 Hours at 5° C. (Table 23).

TABLE 23
Validation Summary of Assay of Sovateltide Injection

| S. | Validation | | |
|---|---|---|---|
| No. | Parameter | Results | Acceptance Criteria |
| 1. | Specificity | No interference is observed | In blank solution |
| | A. Inter- | at retention time of | chromatogram, |
| | ference | Sovateltide peak | there should |
| | Studies | in blank & | not be any peak |
| | | placebo solutions. | interference |
| | | Sovateltide peak | at the retention time of |
| | | was found to be | Sovateltide peak. |
| | | homogeneous and | In the placebo solution |
| | | peak purity is | chromatogram, |
| | | passed in standard | there should |
| | | and sample | not be any peak |
| | | solution. | interference |
| | | | at the retention |
| | | | time of the |
| | | | Sovateltide peak. |
| | | | In the sample solution |
| | | | chromatogram, |
| | | | peak purity |
| | | | of the Sovateltide peak |
| | | | should pass. |
| | B. Force | Based on the observed | All unknown |
| | Degrada- | results, Lyophilized | peaks should |
| | tion | Sovateltide for | be resolved from |
| | Studies | injection was | Sovateltide Peak. |
| | | found sensitive towards | The peak purity of the |
| | | acid, base & peroxide | Sovateltide peak in all |
| | | and photolytic degradation. | stressed samples should |
| | | The chromatograms | pass. |
| | | and peak purity values | Degradation |
| | | of degradation | should not be |
| | | sample solutions indicate | more than 30% in any |
| | | that the analyte peaks | condition. |
| | | are homogeneous | |
| | | and there are no co-eluting | |
| | | peaks from degradation. | |
| | | Sovateltide peak | |
| | | purity in each | |
| | | stress conditions | |
| | | chromatograms | |
| | | were passed. | |
| 2. | Precision | The percentage | The percentage relative |
| | A. System | relative standard | standard deviation |
| | precision | deviation (% RSD) of | (% RSD) of area counts |
| | | area counts of Sovateltide | of Sovateltide peak |
| | | peak from six replicate | from six replicate |
| | | injections of standard | injections of standard |
| | | solution were found | solution should not be |
| | | 1.61% which is within | more than 10.0%. |
| | | acceptance criteria. | |
| | B. Method | The % RSD for | Each sample should meet |
| | Precision | unspecified impurities | the specification. |
| | | above LOQ (0.1%) & | % RSD results for each |
| | | total unspecified | individual |
| | | impurities of six sample | impurity which |
| | | preparations was within | are above limit of |
| | | the acceptance criteria. The | quantification |
| | | study reveals that the test | (0.1%) from |
| | | results obtained by this | six sample preparations |
| | | method are repeatable and | should not be more than |
| | | the method was found | 10.0%. |
| | | to be precise. | |
| | C. Inter- | The % RSD of each | Each sample should meet |
| | mediate | unspecified impurity | the specification. |
| | Precision | above LOQ (0.1%) & | % RSD results for each |
| | & | total unspecified | individual |
| | Rugged- | impurity from | impurity which |
| | ness | six samples were | are above the limit of |
| | | observed within | quantification |
| | | the acceptance criteria. | (0.1%) from |
| | | The overall % RSD of each | six sample preparations |
| | | unspecified | should not be more than |
| | | impurity & total | 10.0%. |
| | | unspecified impurity | Overall % RSD |
| | | of replicate test | of results of |
| | | preparations (12 results) | replicate test preparations |
| | | of method precision and | (12 results) of method |
| | | intermediate precision was | precision and intermediate |
| | | observed within the | precision should not be |
| | | acceptance criteria. | more than 10.0% for each |
| | | The study concludes that | individual impurity which |
| | | the method was found to | are above limit of |
| | | be rugged and precise | quantification (0.1%). |
| | | against variabilities | |
| | | like two different HPLC | |
| | | instruments, analysts, | |
| | | columns, days. | |
| 3. | A. Limit of | The limit of detection for | % RSD of area counts for |
| | Detection | Sovateltide was | Sovateltide |
| | | observed at 0.03% | shall not be more |
| | | with respect to sample | than 33.0% for limit of |
| | | concentration. | detection. |
| | | % RSD at limit of | |
| | | detection was | |
| | | observed 6.62% | |
| | | for Sovateltide, | |
| | | which is within the | |
| | | acceptance criteria. | |
| | B. Limit | The limit of quantitation | % RSD of area counts for |
| | of | for Sovateltide was | Sovateltide shall |
| | Quanti- | observed at 0.101% | not be more |
| | tation | with respect to sample | than 10.0% for limit of |
| | | concentration. | quantitation. |
| | | % RSD at limit | |
| | | of quantitation | |
| | | was observed 3.01% for | |
| | | Sovateltide, which | |
| | | is within the | |
| | | acceptance criteria. | |
| 4. | Linearity | The correlation | Correlation coefficient |
| | | coefficient was | should not be |
| | | observed 0.9998 | less than 0.98. |
| | | for Sovateltide, | For detector |
| | | which was meeting the | linearity the % |
| | | acceptance criteria. | Y-intercept at 100% |
| | | Detector linearity | response level should be |
| | | for Sovateltide | within ±2.0. |
| | | the % Y-intercept at 100% | |
| | | response level | |
| | | was observed at | |
| | | 0.0204, which | |
| | | was meeting the | |
| | | acceptance criteria. | |
| 5. | Accuracy | The method was | Individual % |
| | | found accurate, | recovery and |
| | | and all results were within | mean % recovery at LOQ |
| | | acceptance criteria. | level should be in between |
| | | Individual and overall % | 80.0% and 120.0%. |
| | | recovery, mean % | Individual % |
| | | recovery & % | recovery and |
| | | RSD results were | mean % recovery |
| | | found meeting | at other levels |
| | | the acceptance criteria. | should be in between |
| | | Overall % RSD | 90.0% and 110.0%. |
| | | was found 5.18%. | % RSD should not |
| | | | be more than 10.0% |
| | | | at each level. |
| | | | Overall % RSD should |
| | | | not be more than 10.0%. |
| 6. | Range | From the data of precision, | Acceptance criteria |
| | | linearity, and accuracy, it is | mentioned |
| | | observed that the | under the validation |
| | | method has a range | parameters |
| | | of 0.018 μg/mL to 0.440 | of linearity, accuracy and |
| | | μg/mL for standard of | precision should pass. |
| | | Sovateltide. | |
| | | All acceptance criteria | |
| | | mentioned under the | |
| | | validation parameters of | |
| | | linearity, accuracy | |
| | | and precision | |
| | | were found within | |
| | | acceptance criteria. | |
| 7. | Stability | From the results it is | % Cumulative RSD for |
| | of | concluded that standard is | standard solution |
| | Analytical | stable in solution for up | should not |
| | Solution | to 60 Hours and control | be more than 10.0%. |
| | | unspiked sample is | % Impurity |
| | | stable in solution for up to | above reporting |
| | | 12 Hours at 5° C.. | threshold (0.1%) and |
| | | | below 0.5% should not |
| | | | differ from the |
| | | | initial value |
| | | | by more than ±30.0%. |
| | | | % Impurity above 0.5% |
| | | | should not differ from the |
| | | | initial value by |
| | | | more than ±10.0%. |
| | | | No new peak |
| | | | should appear |
| | | | from the initial sample. |
| 8. | Robustness | The method remains | Should comply with the |
| | | unaffected due to all | system suitability |
| | | deliberate changes to the | parameters as per the test |
| | | analytical method. Hence, | method. |
| | | the method is robust for | RT should be comparable. |
| | | the intended use. | |
| 9. | System | Column efficiency | The column efficiency |
| | Suitability | (USP Plate | (USP plate count) |
| | | Count) for Sovateltide | determined from the |
| | | peak from first injection | Sovateltide peak |
| | | of standard solution from | should not |
| | | each parameter and each | be less than 2000 |
| | | robustness condition | theoretical plates |
| | | were found within | from first |
| | | the acceptance criteria. | injection of standard |
| | | The % relative standard | solution. |
| | | deviation of area counts | The percentage relative |
| | | of Sovateltide peak | standard deviation |
| | | from three replicate | (% RSD) |
| | | injections of standard | of area counts of |
| | | solution from each | Sovateltide peak |
| | | parameter and | from three |
| | | each robustness | replicate injections of |
| | | condition were | standard solution |
| | | found within the | should not |
| | | acceptance criteria. | be more than 10.0%. |

### Related Substances Method Validation Summary:

Chromatographic Parameters for method validation of Related Substance of Sovateltide Injection (Method 1): Method-1 utilizes chromatographic conditions to separate and quantify a specific compound of interest. The analytical column used is either the Xtimate C18 Column or the ChromCore 120 C18 Column, both with dimensions of 4.6×250 mm and 5 m. A Ghost Buster Column with dimensions of 4.6×50 mm is used to detect any unexpected peaks, which are usually called ghost peaks that may interfere with the results. The flow rate is set to 0.5 mL/min, with an injection volume of 90 μL. The detection wavelength is 215 nm, and the auto sampler temperature is maintained at 5° C. The column oven temperature is set to 50° C., and the run time for the method is 120 min. The retention time for the compound of interest is approximately 66.0 min under these chromatographic conditions. These parameters ensure the separation and accurate quantification of the compound in the sample.

### Mobile Phase for Un-Specified Impurities (Method 1):

### Mobile Phase A (KH2PO4+Sodium Chloride in HPLC Water).

### Mobile Phase-B (Methanol).

Referring to Table 24, a tabular representation of a Method Validation Summary of Related Substance in Sovateltide Injection (Method 1) is illustrated. After evaluation of data, the method was found precise, linear, accurate, robust, rugged and specific. As the results of all the validation parameters were within the acceptance criteria, it is concluded that the analytical method is suitable for the determination of organic impurities (unspecified) in Lyophilized Sovateltide for Injection by HPLC. Based on solution stability study, standard solution was stable up to 60 Hours and sample solution was stable up to 12 Hours at 5° C. (Table 24).

TABLE 24
Method Validation Summary of Related Substance in Sovateltide Injection
(Method 1)

| S. | Validation | | |
|---|---|---|---|
| No. | Parameter | Results | Acceptance Criteria |
| 1. | Specificity | No interference is | In blank solution |
| | A. Inter- | observed at retention | chromatogram, there should |
| | ference | time of Sovateltide | not be any peak interference |
| | Studies | peak in blank & placebo | at the retention time of the |
| | | solutions. | Sovateltide peak. |
| | | Sovateltide peak | In the placebo solution |
| | | was found to | chromatogram, there should |
| | | be homogeneous and | not be any peak interference |
| | | peak purity is passed | at the retention time of the |
| | | in standard and | Sovateltide peak. |
| | | sample solution. | In the sample solution |
| | | | chromatogram, peak purity |
| | | | of the Sovateltide peak |
| | | | should pass. |
| | B. Force | Based on the observed | All unknown peaks should |
| | Degra- | results, Lyophilized | be resolved |
| | dation | Sovateltide for | from Sovateltide |
| | Studies | injection was found | Peak. |
| | | sensitive towards acid, | The peak purity of |
| | | base & peroxide and | Sovateltide peak in all |
| | | photolytic degradation. | stressed samples should |
| | | The chromatograms | pass. |
| | | and peak purity values | Degradation should not be |
| | | of degradation | more than 30% in any |
| | | sample solutions | condition. |
| | | indicate that | |
| | | the analyte peaks are | |
| | | homogeneous | |
| | | and there are no | |
| | | co-eluting peaks from | |
| | | degradation. | |
| | | Sovateltide peak | |
| | | purity in each | |
| | | stress conditions | |
| | | chromatograms | |
| | | were passed. | |
| 2. | Precision | The percentage | The percentage |
| | D. System | relative standard | relative standard |
| | precision | deviation (% RSD) of | deviation (% RSD) |
| | | area counts of Sovateltide | of area counts of |
| | | peak from six | Sovateltide peak from |
| | | replicate injections of | six replicate injections of |
| | | standard solution were | standard solution |
| | | found 1.61% which | should not be |
| | | is with in acceptance | more than 10.0%. |
| | | criteria. | |
| | E. Method | The % RSD | Each sample should |
| | Precision | for unspecified | meet the specification. |
| | | impurities above | % RSD results for each |
| | | LOQ (0.1%) & | individual impurity which |
| | | total unspecified | are above limit of |
| | | impurities of six | quantification (0.1%) from |
| | | sample preparations | six sample preparations |
| | | was within | should not be |
| | | the acceptance | more than 10.0%. |
| | | criteria. The study | |
| | | reveals that the test | |
| | | results obtained by this | |
| | | method are repeatable | |
| | | and the method was | |
| | | found to be precise. | |
| | F. Inter- | The % RSD of each | Each sample should |
| | mediate | unspecified | meet the specification. |
| | Precision | impurity above | % RSD results for each |
| | & | LOQ (0.1%) & total | individual impurity which |
| | Rugged- | unspecified impurity | are above limit of |
| | ness | from six samples | quantification (0.1%) |
| | | were observed within | from six sample |
| | | the acceptance criteria. | preparations should not |
| | | The overall % RSD | be more than 10.0%. |
| | | of each unspecified | Overall % RSD |
| | | impurity & total | of results of |
| | | unspecified impurity of | replicate test |
| | | replicate test | preparations |
| | | preparations (12 results) | (12 results) of method |
| | | of method precision | precision and intermediate |
| | | and intermediate | precision should |
| | | precision was | not be more |
| | | observed within the | than 10.0% for each |
| | | acceptance criteria. | individual impurity which |
| | | The study concludes | are above limit of |
| | | that the method | quantification (0.1%). |
| | | was found to be | |
| | | rugged and precise | |
| | | against variabilities like | |
| | | two different HPLC | |
| | | instruments, analysts, | |
| | | columns, days. | |
| 3. | A. Limit | Limit of detection for | % RSD of area counts for |
| | of | Sovateltide was observed | Sovateltide shall |
| | Detection | at 0.03% with respect | not be more |
| | | to sample concentration. | than 33.0% for limit of |
| | | % RSD at limit of | detection. |
| | | detection was observed | |
| | | 6.62% for Sovateltide, | |
| | | which is within | |
| | | the acceptance criteria. | |
| | C. Limit | Limit of quantitation for | % RSD of area counts for |
| | of | Sovateltide was observed | Sovateltide shall |
| | Quan- | at 0.101% with respect | not be more |
| | titation | to sample concentration. | than 10.0% for limit of |
| | | % RSD at limit | quantitation. |
| | | of quantitation | |
| | | was observed 3.01% for | |
| | | Sovateltide, | |
| | | which is within | |
| | | the acceptance criteria. | |
| 4. | Linearity | Correlation coefficient | Correlation coefficient |
| | | was observed 0.9998 for | should not be less than 0.98. |
| | | Sovateltide, which was | For detector linearity the % |
| | | meeting the acceptance | Y-intercept at 100% |
| | | criteria. | response level should be |
| | | Detector linearity for | within ±2.0. |
| | | Sovateltide the | |
| | | % Y-intercept | |
| | | at 100% response | |
| | | level was observed at | |
| | | 0.0204, which was | |
| | | meeting the acceptance | |
| | | criteria. | |
| 5. | Accuracy | The method was found | Individual % recovery and |
| | | accurate, and all | mean % recovery at LOQ |
| | | results were | level should be in between |
| | | within acceptance criteria. | 80.0% and 120.0%. |
| | | Individual and overall % | Individual % recovery and |
| | | recovery, mean | mean % recovery at other |
| | | % recovery & | levels should be in between |
| | | % RSD results were found | 90.0% and 110.0%. |
| | | meeting the acceptance | % RSD should not be more |
| | | criteria. | than 10.0% at each level. |
| | | Overall % RSD was found | Overall % RSD should not |
| | | 5.18% | be more than 10.0%. |
| 6. | Range | From the data of | Acceptance criteria |
| | | precision, linearity, | mentioned under the |
| | | and accuracy, it is | validation parameters |
| | | observed that the | of linearity, accuracy and |
| | | method has a range of | precision should pass. |
| | | 0.018 μg/mL to 0.440 | |
| | | μg/mL for standard of | |
| | | Sovateltide. | |
| | | All acceptance criteria | |
| | | mentioned under | |
| | | the validation | |
| | | parameters of linearity, | |
| | | accuracy and | |
| | | precision were | |
| | | found within acceptance | |
| | | criteria. | |
| 7. | Stability of | From results it is | % Cumulative RSD for |
| | Analytical | concluded that | standard solution should |
| | Solution | standard is stable | not more than 10.0%. |
| | | in solution for | % Impurity above reporting |
| | | up to 60 hrs. and | threshold (0.1%) and below |
| | | control unspiked | 0.5% should not differ from |
| | | sample is stable in | initial value by more than |
| | | solution for up | ±30.0%. |
| | | to 12 Hours at 5° C.. | % Impurity above 0.5% |
| | | | should not differ from initial |
| | | | value by more than ±10.0%. |
| | | | No new peak should appear |
| | | | from the initial sample. |
| 8. | Robust- | The method remains | Should comply with the |
| | ness | unaffected due to all | system suitability |
| | | deliberate changes to | parameters |
| | | the analytical method. | as per the test method. |
| | | Hence, the method | RT should be |
| | | is robust for the | comparable. |
| | | intended use. | |
| 9. | System | Column efficiency | The column efficiency |
| | Suitability | (USP Plate Count) for | (USP plate count) |
| | | Sovateltide peak | determined from |
| | | from first injection | Sovateltide peak |
| | | of standard solution | should not |
| | | from each parameter | be less than 2000 |
| | | and each | theoretical plates from |
| | | robustness condition | first injection of |
| | | were found within the | standard solution. |
| | | acceptance criteria. | The percentage relative |
| | | The % relative standard | standard deviation |
| | | deviation of area counts | (% RSD) of area |
| | | of Sovateltide peak from | counts of Sovateltide |
| | | three replicate injections | peak from three replicate |
| | | of standard solution | injections of standard |
| | | from each parameter | solution should |
| | | and each robustness | not be more |
| | | condition | than 10.0%. |
| | | were found within the | |
| | | acceptance criteria. | |

Chromatographic Parameters for method validation of Related Substance of Sovateltide Injection (Method 2): The given information describes the conditions and parameters used in an analytical column for a chromatographic analysis. The Ultisil XB-C30 Column or ChromCore C30 Column with dimensions of 4.6×250 mm and a particle size of 5 m is used as the analytical column. The Ghost-Buster Column with dimensions of 4.6×50 mm is used for removing. The flow rate is maintained at 0.5 mL/min, and an injection volume of 80 μL is used. The detection wavelength is set to UV at 215 nm, and the autosampler temperature is maintained at 5° C. while the column oven temperature is set at 50° C. The standard run time is 30 min, while it is 80 min for sample solutions. The retention time for the analysis is between 6 to 12 minutes, and the relative retention time is approximately 0.2 with respect to Sovateltide. These parameters are critical to obtaining accurate and reproducible results in a chromatographic analysis.

Mobile Phase for Specified Impurities (Method 2): Mobile Phase A (0.05 M 1-Octane Sulfonic acid sodium salt, pH 7.5):

### Mobile Phase-B (Methanol).

Referring to Table 25, a tabular representation of a Validation Summary of Related Substance in Sovateltide Injection (Method 2) is illustrated. After evaluation of data, the method was found precise, linear, accurate, robust, rugged and specific. As the results of all the validation parameters were within the acceptance criteria, it is concluded that the analytical method is suitable for the determination of organic impurities (Specified—D-His) in Lyophilized Sovateltide for Injection by HPLC. Based on solution stability study, standard solution was stable up to 48 Hours, spiked sample solution was stable up to 40 hrs and control sample solution was stable up to 39 Hours at 5° C. (Table 25).

TABLE 25
Validation Summary of Related Substance in Sovateltide
Injection (Method 2)

| S. | Validation | | |
|---|---|---|---|
| No. | parameter | Results | Acceptance Criteria |
| 1. | Specificity | No interference | In blank solution |
| | A. Inter- | is observed | chromatogram, there |
| | ference | at retention | should not be any peak |
| | Studies | time of D-His | interference at the |
| | | peak. | retention time of |
| | | D-His peak | D-His & Sovateltide |
| | | was found to | peak. |
| | | be homogeneous | In Placebo solution |
| | | and peak | chromatogram, there |
| | | purity is passed | should not be any |
| | | in standard | peak interference at the |
| | | & spike | retention time of D-His |
| | | sample solution. | & Sovateltide |
| | | Sovateltide peak | peak. |
| | | was found | In Main analyte |
| | | to be homogeneous | (Sovateltide) solution |
| | | and peak purity | chromatogram, there |
| | | is passed in | should not be any |
| | | Main analyte | peak |
| | | solution & | interference at the |
| | | sample and | retention time of |
| | | spike sample | D-His peak. |
| | | solution. | In Sample solution |
| | | | chromatogram, peak |
| | | | purity of D-His peak |
| | | | & Sovateltide peak |
| | | | should pass. |
| | | | In Spike sample |
| | | | solution chromatogram, |
| | | | peak purity of D-His |
| | | | peak & Sovateltide |
| | | | peak should pass. |
| | Force | Sovateltide peak purity in | No interference |
| | Degra- | each stress conditions | should be observed from |
| | dation | chromatograms were | placebo or diluent |
| | Studies | passed. | at retention time of D- |
| | | The study concludes that | His Peak. |
| | | the related substances | Peak purity of |
| | | method is specific. | Sovateltide peak in all |
| | | All peaks are well resolved | stressed samples |
| | | from Sovateltide peak. | Peak purity of |
| | | | D-Hisshould pass. |
| | | | peak in all stressed |
| | | | samples should pass. |
| | | | Degradation should |
| | | | not be more than 30% |
| | | | in any condition. |
| | Precision | The percentage relative | The percentage relative |
| | G. System | standard deviation (% RSD) | standard deviation (% |
| | precision | of area counts of D-His peak | RSD) of area counts |
| | | from six replicate injections | of D-His peak from six |
| | | of standard solution were | replicate injections |
| | | found 1.44% which is with in | of standard solution |
| | | acceptance criteria. | should not be more |
| | | | than 5.0%. |
| | H. Method | In Unspiked (Control) | Each sample should |
| | Precision | sample no peak was found | meet the specification. |
| | | at the RT of D-His. | % RSD of results |
| | | The % RSD of D-His of | of six sample |
| | | six spike sample | preparations should |
| | | preparations was observed | not be more than 10.0%. |
| | | 0.30%, which is within the | |
| | | acceptance criteria. | |
| 2. | I. Inter- | The % RSD of D-His (%) | Each sample should |
| | mediate | from six samples was | meet the specification. |
| | Precision | observed 6.17%, which is | % RSD results for |
| | | within the acceptance | D-His from six spike |
| | | criteria. Data indicates that | sample preparations |
| | | the method has an | should not be more |
| | | acceptable level of | than 10.0%. |
| | | precision. | Overall % RSD of |
| | | The overall % RSD of D- | results of replicate test |
| | | His of replicate test | preparations (12 |
| | | preparations (12 results) of | results) of method |
| | | method precision and | precision and |
| | | intermediate precision was | intermediate precision |
| | | observed 4.17%, which is | should not be more |
| | | meeting the acceptance | than 10.0% for D-His. |
| | | criteria. | |
| 3. | A. Limit of | Limit of detection for D- | % RSD of area |
| | Detection | His was observed at 0.06% | counts for specified |
| | | with respect to sample | impurity (D-His) |
| | | concentration. | shall not be more than |
| | | % RSD at limit of | 33.0% for limit |
| | | detection were observed | of detection. |
| | | 9.40 for D-His, which is | |
| | | within the acceptance | |
| | | criteria. | |
| | D. Limit | Limit of quantitation for D- | % RSD of area |
| | of | His was observed at 0.18% | counts for specified |
| | Quanti- | with respect to sample | impurity (D-His) |
| | tation | concentration. | shall not be more than |
| | | % RSD at limit of | 10.0% for limit |
| | | quantitation were observed | of quantitation. |
| | | 5.75% for D-His, which is | |
| | | within the acceptance | |
| | | criteria. | |
| 4. | Linearity | Correlation coefficient was | Correlation coefficient |
| | | observed 0.9996 for D-His, | should not be less |
| | | which was meeting the | than 0.98. |
| | | acceptance criteria. | For detector linearity |
| | | Detector linearity for | the % Y-intercept at |
| | | Sovateltide the % Y- | 100% response level |
| | | intercept at 100% response | should be within ±2.0. |
| | | level was observed at | |
| | | 0.0401 and for D-His | |
| | | which was meeting the | |
| | | acceptance criteria. | |
| 5. | Accuracy | The method was found | Individual % recovery |
| | | accurate, and all results | and mean % |
| | | were within acceptance | recovery at LOQ |
| | | criteria. | level should be in |
| | | Individual and overall % | between 80.0% |
| | | recovery, mean % recovery | and 120.0%. |
| | | & % RSD results were | Individual % |
| | | found meeting the | recovery and mean % |
| | | acceptance criteria. | recovery at other |
| | | Overall % RSD for D-His | levels should be in |
| | | was observed 8.18. | between 90.0% |
| | | | and 110.0%. |
| | | | % RSD should |
| | | | not be more |
| | | | than 10.0% at each level. |
| | | | Overall % RSD should |
| | | | not be more than 10.0%. |
| 6. | Range | From the data of precision, | Acceptance criteria |
| | | linearity, and accuracy, it is | mentioned under the |
| | | observed that the method | validation parameters |
| | | has a range d 0.036 μg/mL | of linearity, |
| | | to 0.401 μg/mL for D-His. | accuracy and |
| | | All acceptance criteria | precision should pass. |
| | | mentioned under the | |
| | | validation parameters of | |
| | | linearity, accuracy and | |
| | | precision were found within | |
| | | acceptance criteria. | |
| 7. | Stability of | From results it is concluded | % Cumulative RSD |
| | Analytical | that standard is stable in | for standard solution |
| | Solution | solution for up to 48 Hours, | should not be |
| | | spiked sample is stable in | more than 5.0%. |
| | | solution for up to 40 Hours | % Impurity above |
| | | and control unspiked sample | 0.5% should not differ |
| | | is stable in solution for up to | from initial value by |
| | | 39 Hours at 5° C.. | more than ±10.0%. |
| 8. | Robustness | The method remains | Should comply with |
| | | unaffected due to all | the system suitability |
| | | deliberate changes to the | parameters as per |
| | | analytical method. | the test method. |
| | | Hence, the | RT & RRT should |
| | | method is robust for the | be comparable. |
| | | intended use. | |
| 9. | System | The tailing factor for D- | The tailing factor |
| | Suitability | His peak from first | determined from D-His |
| | | injection of standard | peak should not more |
| | | solution from each | than 3.0 from first |
| | | parameter and each | injection of standard |
| | | robustness condition was | solution. |
| | | found within the | The percentage relative |
| | | acceptance criteria. | standard deviation |
| | | The % relative standard | (% RSD) of area |
| | | deviation of area counts of | counts of Sovateltide |
| | | D-His peak from six | peak from three |
| | | replicate injections of | replicate injections of |
| | | standard solution from | standard solution s |
| | | each parameter and each | hould not be more than |
| | | robustness condition was | 10.0%. |
| | | found within the | |
| | | acceptance criteria. | |

Although the field of the invention has been described herein with limited reference to specific embodiments, this description is not meant to be construed in a limiting sense. Various modifications of the disclosed embodiments, as well as alternate embodiments of the invention, will become apparent to persons skilled in the art upon reference to the description of the invention.

We claim:

1. A lyophilized Sovateltide-based injectable formulation, comprising:
i) an active pharmaceutical ingredient in the range of 0.01-0.02% w/w;
ii) at least two soluble excipients in the range of 20-80% w/w; and
iii) water for injection in the range of 1-2% w/w.

2. The formulation as claimed in claim 1, wherein said active pharmaceutical ingredient is Sovateltide.

3. The formulation as claimed in claim 1, wherein said soluble excipient includes, but not limited to mannitol, Trisodium citrate Dihydrate, Citric Acid anhydrous, Dibasic sodium citrate, Dibasic sodium Phosphate, Sodium Chloride, Hydroxypropyl beta cyclodextrin.

4. The formulation as claimed in claim 1, wherein said soluble excipients are ideally mannitol as freeze-drying filler and Trisodium Citrate Dihydrate as buffering agent.

5. A method for preparation of said lyophilized Sovateltide-based injectable formulation as claimed in claim 1, the method comprising the steps:
a) dissolving said Trisodium Citrate Dihydrate in said water for injection in order to obtain a mixture;
b) adding said active pharmaceutical ingredient in said mixture, followed by addition of said Mannitol in order to obtain a solution;
c) checking the pH of said solution for making up the volume of said solution, followed by sterile filtering said solution and filling said sterile filtered solution in vials; and
d) half stoppering of plugs on said vials, followed by loading said vials in freeze dryer for lyophilization to obtain said formulation.

6. The method as claimed in claim 5, wherein the vials filled with the filtered solution are subjected to lyophilization by freezing at −45° C. and then drying at −20° C., at 0° C. and then at +10° C.

7. The method as claimed in claim 5, wherein said vials are unloaded from said freeze dryer and sealed to obtain said lyophilized Sovateltide-based injectable formulation.

8. The method as claimed in claim 5, wherein said formulation is administered through intravenous route.

9. The method as claimed in claim 5, wherein said vials are washed and depyrogenated before filling said sterile filtered solution in said vials.

10. The method as claimed in claim 5, wherein said lyophilized Sovateltide-based injectable formulation is stored at 2-8 degree Celsius or 22° C. to 28° C.
